(19) United States
(12) Patent Application Publication
(10) Pub. No.: US 2015/0223743 A1
(43) Pub. Date: Aug. 13, 2015

(54) METHOD FOR MONITORING A HEALTH CONDITION OF A SUBJECT

(71) Applicant: Wipro Limited, Bangalore (IN)

(72) Inventors: Vinod Pathangay, Bangalore (IN); Satish Prasad Rath, Bangalore (IN)

(73) Assignee: Wipro Limited, Bangalore (IN)

(21) Appl. No.: 14/230,694

(22) Filed: Mar. 31, 2014

(30) Foreign Application Priority Data
Feb. 12, 2014 (IN) 662/CHE/2014

(51) Int. Cl. A61B 5/18; A61B 5/0476; A61B 5/0488; A61B 5/00; A61B 5/0205; A61B 5/0295; A61B 5/04; A61B 5/0402; A61B 5/026

(52) U.S. Cl. CPC A61B 5/18; A61B 5/0402; A61B 5/0476; A61B 5/0488; A61B 5/0261; A61B 5/0205; A61B 5/0295; A61B 5/04015; A61B 5/6824; A61B 5/6828; A61B 5/6823; A61B 5/6816; A61B 5/6826; A61B 5/7246; A61B 5/742

(57) ABSTRACT

Embodiments of the present disclosure provide a method and a computing unit to monitor health condition of a subject. The computing unit receives physiological signals from a plurality of sensors placed on the subject. The computing unit detects a work-type based on the physiological signals received from the plurality of sensors and assigns a weight to each of the plurality of sensors based on the work-type. Thereafter, the computing unit generates a fatigue score using the physiological signals and the weight of the plurality of sensors. The fatigue score indicates the health condition of the subject.

This application claims the benefit of Indian Patent Application Filing No. 662/CHE/2014, filed Feb. 12, 2014, which is hereby incorporated by reference in its entirety.

### FIELD OF THE INVENTION

Embodiments of the present disclosure relate to monitoring physiological signals of a subject. More particularly, the present disclosure relates to monitoring health condition of a subject using physiological signal of a subject.

### BACKGROUND OF THE INVENTION

Presently, shift work typically involves workers (alternatively referred as operators) working for various amounts of time throughout a day. Work shifts may occur at any time during the day, and are often not synchronized with the natural sleep and waking patterns of those who work in the shifts. Assessing fatigue due to shift work or for any other reason has historically been a subjective, narrow effort, because of the non-existence of fatigue accessing or management systems. Generally, many managers neither acknowledge nor take adequate preemptive steps to mitigate risks due to excessive fatigue of the workers. Moreover, while workers may recognize their own fatigue at some level, they often underestimate dangerous fatigue levels due to lack of knowledge or negligence and engage in habits that promote unnecessary safety risks.

The shift work affects millions of shift workers by the risk of fatigue. The shift workers' generally have partial poorer health due to a lack of proper real time fatigue management systems, which may improve the health conditions of the shift workers. The shift work is not limited to physical work; it may include mental activity as well. Society is affected by way of fatigue-related driving fatalities and accidents because of lack of vigilance among shift workers performing safety-critical tasks. Examples of catastrophic fatigue-related errors and accidents abound, such as accidents directly caused by fatigued workers e.g. air traffic controllers, rail engineers, bus drivers and accidents caused indirectly due to fatigue. Besides safety, the economic costs to consumers, governments, and companies due to fatigue are staggering, numbering in the billions of dollars annually worldwide.

There exist devices which allow the monitoring of current alertness levels of an operator. For example, a grip-responsive operator alertness monitor includes a pressure sensor associated with a mechanism for controlling a vehicle. The pressure sensor detects operator fatigue as exhibited by a change in operator pressure on the control mechanism. An operator stimulus is coupled to the pressure sensor and, upon sensing fatigue, produces a stimulus such as a visual or audible alarm. In another example a device determines whether an eye within a field of view is closed for a predetermined period of time. If so, the assumption is made that the subject has fallen asleep, so that corrective measures can be taken, such as the sounding of an alarm. All of the above-referenced devices are designed to monitor current alertness level. None of them predict risk in any way, nor do they determine the level of risk or address countermeasures based on risk level.

Further, the risk of fatigue level not only occurs with workers working in physically challenged environment, but it also affects workers working in non-physically challenged environments such as offices. In the recent past, with the pervasiveness of computer-human information exchange employees/workers spend hours interacting with computers using input devices such as keyboards and computer mice and viewing information output on computer displays includes, but not limited to, cathode ray tube (CRT), light emitting diode (LED), and liquid crystal display (LCD) technologies. Cognitive and visual fatigue resulting from repetitive task execution and long hours of viewing electronic displays will impact on efficiency of the employees/workers.

In an effort to detect and manage the impact of cognitive and eye fatigue, various devices and methods of detection and control have been proposed. One of such devices measures and evaluates eye activity based on pupil diameter and position, visual fixation frequency and duration, and blink frequency, and applies them to alertness models to determine onset of user fatigue or drowsiness in real-time. Another device with fixed-plane focus results in eye-strain which can be relieved by periodic use of eye exercises where the user focuses on 3-dimensional images or lights placed in multiple planes of focus. Another device uses a 3-dimensional air sculpture that relieves eyestrain by allowing a user to focus attention on the sculpture. Also, the device allows the user to exercise the eyes by periodically following a series of lights placed at various positions in 3-dimensional space. The described prior-art references, however, do not examine manual task performance as a tool for detection of cognitive and visual fatigue. One such tool would examine degradation in proficiency at entering data using a keyboard, touch screen, joystick and/or mouse as an indicator of user fatigue.

Though the advance in the technology has mechanized a lot of work in the industrial sector but still major part of workers in transportation, security, production, construction, mining and other related industries perform their duties in day-night shifts in order to facilitate round-the-clock business operations. This inherently leads to workers getting exhausted due to long work hours and the nature of working against their biological rhythms. Also, there are possibilities of double-shifts or travelling longer distances where a worker may extend number of hours of duty without taking adequate rest and relaxation. This may cause a gradually increasing level of physical and mental exhaustion, which may be the reason for fatigue. It may be difficult for the supervisor to manage workers in his team and ensure safety of all the workers because of fatigue. There are no means to help supervisor to utilize his resources and take a decision so that none of the workers get exhausted and avoid chances of burn out or accidents.

At present, there exists no system or means of warning the worker and/or the worker's employer of over-working an individual to the point of being a danger to themselves and those around them. There is no system available that helps both the worker and the employer understand in real-time the accumulative fatigue condition of the worker over a given period of time. For example, a person may work a normal twelve hour night shift from 7:00 PM to 7:00 AM and may then be asked to work another four hours by his supervisor, as a replacement worker. After the continuous sixteen hours of work, the worker leaves may turn to work the next day nearly taking minimal rest. In most cases neither the worker, nor the worker's supervisor or manager, is aware of the risk which is accumulated because of the continuous work that could make the worker and/or the workplace in jeopardy. There is no system or method currently available to warn the worker or the employer of a potential fatigue-related problem.

Accordingly, a need exists for a method and system which provides a means to perceive fatigue, identify and implement appropriate countermeasures. Thereby, improving safety, health condition and performance of workers, especially shift workers.

### SUMMARY OF THE INVENTION

The shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

In one non-limiting embodiment, the present disclosure provides a method for monitoring health condition of a subject. The method comprises receiving by a computing unit, physiological signals from a plurality of sensors placed on the subject. The computing unit detects a work-type based on the physiological signals from the plurality of sensors. Upon detecting the work type, the computing unit assigns a weight to each of the plurality of sensors based on the work-type. Thereafter, the computing unit generates a fatigue score using the physiological signals and the weight of the plurality of sensors. The fatigue score indicates the health condition of the subject.

In one non-limiting embodiment, the present disclosure also provides a computing unit to monitor health condition of a subject. The computing unit comprises at least one processor and a memory storing instructions executable by the at least one processor, wherein the instructions configure the at least one processor to receive physiological signals from a plurality of sensors placed on the subject, detect a work type based on the physiological signals, assign a weight to each of the plurality of sensors based on the work type and generate a fatigue score using the physiological signals and the weight of the plurality of sensors, wherein the fatigue score indicates the health condition of the subject.

In one non-limiting embodiment, the present disclosure further provides a non-transitory computer readable medium including operations stored thereon that when processed by at least one processor cause a system to perform the acts of receiving physiological signals from a plurality of sensors placed on the subject, detecting a work-type based on the physiological signals, assigning a weight to each of the plurality of sensors based on the work-type, and generating a fatigue score using the physiological signals and the weight of the plurality of sensors, wherein the fatigue score indicates the health condition of the subject.

The foregoing summary is illustrative only and is not intended to be in any way limiting. In addition to the illustrative aspects and features described above, further aspects, and features will become apparent by reference to the drawings and the following detailed description.

### BRIEF DESCRIPTION OF THE DRAWINGS

The novel features and characteristic of the disclosure are set forth in the appended claims. The embodiments of the disclosure itself, however, as well as a preferred mode of use, further objectives and advantages thereof, will best be understood by reference to the following detailed description of an illustrative embodiment when read in conjunction with the accompanying drawings. One or more embodiments are now described, by way of example only, with reference to the accompanying drawings.

FIG. 1A illustrates a block diagram of an exemplary computing unit to monitor health condition of a subject in accordance with some embodiments of the present disclosure;

FIG. 2A illustrates a block diagram of an exemplary computing unit to monitor health condition of a subject and display the fatigue score on an associated display in accordance with some embodiments of the present disclosure;

FIG. 2B illustrates a block diagram of an exemplary computing unit to monitor health condition of a subject and an associated display unit for displaying health condition of a subject in accordance with some embodiments of the present disclosure;

FIG. 3A illustrates an environment in which a computing unit receives physiological signals associated with a subject in accordance with some embodiments of the present disclosure;

FIG. 3B illustrates an environment in which a computing unit receives physiological signals from a plurality of subjects in accordance with some embodiments of the present disclosure;

FIG. 4A is a fatigue chart illustrating representation of a fatigue level of a subject in accordance with some embodiments of the present disclosure;

FIG. 4B is a fatigue chart illustrating representation of a fatigue level of a plurality of subjects in accordance with some embodiments of the present disclosure;

FIG. 5A illustrates an exemplary environment in which health condition of a human is monitored using an exemplary computing unit in accordance with an example embodiment of the present disclosure;

FIG. 5B illustrates an exemplary environment in which health condition of an animal is monitored using an exemplary computing unit in accordance with an example embodiment of the present disclosure; and

FIG. 6 shows a flowchart illustrating a method of monitoring health condition of a subject using a computing device in accordance with some embodiments of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION OF THE INVENTION

The foregoing has broadly outlined the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific aspect disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

Embodiments of the present disclosure relate to monitoring physiological signals of a subject. More particularly, a method for monitoring health condition of a subject using the physiological signals is disclosed. The subject may be one of human being and animal. A plurality of sensors may be placed on the subject at various locations selected from at least one of head, muscles of arms, muscle of legs, scalp, sternum, mid-axillary line, anterior axillary line, ear lobes and finger tips. The method of monitoring health condition of a subject comprises receiving by a computing unit, physiological signals from a plurality of sensors placed on the subject. The computing unit may then detect a work-type based on the physiological signals received from the plurality of sensors. Upon detecting the work type, the computing unit may assign a weight to each of the plurality of sensors based on the work-type. Thereafter, the computing unit may generate a fatigue score using the physiological signals and the weights assigned to the plurality of sensors. The fatigue score may indicate the health condition of the subject. The computing unit may be any device which comprises at least one processor and a memory storing instructions executable by the at least one processor.

The term “health condition” includes, but not limited to fatigue of the subject. The term “fatigue” in ordinary describes a very common phenomenon. For purpose of this disclosure “fatigue” comprises and may be defined as: —awareness of a decreased capacity for physical and/or mental activity due to an imbalance in the availability, utilization, and/or restoration of resources needed to perform activity—a state of weariness related to reduced motivation a transitional state between wakefulness and sleep physical state of disturbed homeostasis due to work or stress, which manifest in loss in efficiency and a general disinclination to work—a feeling of weariness and inability to mobilize energy Onset of fatigue is associated with increased anxiety, decreased short term memory, slowed reaction time, decreased work efficiency, reduced motivational drive, decreased vigilance, increased variability in work performance, increased errors and omissions which increase when time pressure, diminishing of information processing and sustained attention. The term “fatigue” used in the disclosure may be understood to comprise also any term mentioned below so for purposes of this disclosure. Following terms characterizing fatigue may be considered as synonyms. They are: exhaustion, lack of motivation, tiredness, boredom, sleepiness, feeling tired and listless, apathy, indifference, inertia, lethargy, stolidity, vacancy, drowsiness, depletion, feeling weary, feeling tired, strained or sleepy, being tired, being sleepy, being drained, being worn out, being spent, overworked. Also, fatigue can be suitably understood as opposite to following terms: vigilance, alertness, watchfulness, and wakefulness. Any of these terms as for example lack of vigilance, lack of alertness, can be also suitably treated as replacement of word fatigue in accordance with this disclosure.

Henceforth, embodiments of the present disclosure are explained with the help of exemplary diagrams and one or more examples. However, such exemplary diagrams and examples are provided for the illustration purpose for better understanding of the present disclosure and should not be construed as limitation on scope of the present disclosure.

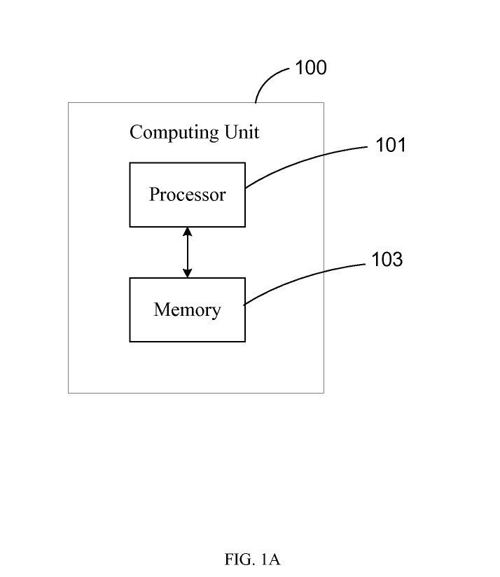

FIG. 1 illustrates an exemplary computing unit 100 adopted for monitoring health condition of a subject in accordance with some embodiments of the present disclosure. The computing unit 100 may include at least one central processing unit (“CPU” or “processor”) 101 and a memory 103 storing instructions executable by the at least one processor. The instructions configure the processor 101 to receive physiological signals from a plurality of sensors placed on the subject. The subject may be one of human being and animal.

The processor 101 may comprise at least one data processor for executing program components for executing user- or system-generated requests. A user may include a person, a person using a device such as such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. The processor 101 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

The sensors may include, but are not limited to, Electrocardiography (ECG) sensor, Electroencephalography (EEG) sensor, Electromyography (EMG) sensor and photo-plethysmo-graphy (PPG) sensor. Initially, the processor 101 may detect a work type based on the physiological signals. Also, the processor 101 assigns a weight to each of the plurality of sensors based on the work type. Thereafter, the processor 101 may generate a fatigue score using the physiological signals and the weight of the plurality of sensors. The fatigue score indicates the health condition of the subject.

The processor 101 may extract frequency domain values from the physiological signals received from the plurality of sensors placed on the subject. Next, the processor 101 may compare the frequency domain values with a plurality of predefined reference values to identify a matching reference value. Thereafter, the processor 101 may identify a work type corresponding to the frequency domain value, which is substantially near to or equal to the matched reference value. After identifying the work type, the processor 101 may assign a weight to each of the plurality of sensors based on the work type.

In some example embodiment of the present disclosure, five sensors, an ECG sensor and four EMG sensors, may be placed on the body of a worker. The processor 101 may receive physiological signals from all the EMG sensors, on which frequency domain analysis is performed to obtain [a₁, a₂, a₃, a₄], where a₁, a₂, a₃, a₄ are electrical signals generated by muscle cells. The EMG sensors may be placed on the muscles of hands and legs of the worker. Also, the processor 101 may receive heart rate signal (h₁) from the ECG sensors, which are placed on either side of the heart. The processor 101 may then generate an input vector v=[aₗ, a₂, a₃, a₄, h₁], using the received data from the all the sensors.

C₁, C₂ and C₃ may be trained reference classifier models for different work types such as walking, driving and load lifting respectively. The trained reference classifier models may be stored in the memory of the computing device 100. The processor 101 generates an output for each reference model by performing predefined computations on the input vector. The outputs y₁, y₂ and y₃ are generated for the reference models C₁, C₂ and C₃ respectively and are represented as,

y₁=C₁(v),

y₂=C₂(v),

y₃=C₃(v).

The processor 101 identifies the work type for the activity which has the highest output value. For example, if the input vector v is [20, 22, 70, 65, 40] based on the signals received from the five sensors placed on the worker, the processor 101 generates the outputs y₁, y₂ and y₃ using the input vector v and the trained reference classifier models as mentioned above. If the values of y1, y2, and y3 are 70, 35 and 20 respectively, the processor compares the output values to determine y₁ has the highest value and subsequently identifies the work type corresponding to y₁ i.e. walking.

Table 1 illustrates work assigned for the received sensor signals based on the reference models, in accordance with the above example.

TABLE 1

| Sensor 1 | Sensor 2 | | Output of | |
|---|---|---|---|---|
| signals | signals | | Reference | |
| (4 EMG | (ECG | | Models | Work |
| sensors) | sensor) | Input vector v | (C1, C2, C3) | type |
| [20, 22, 70, 65] | 40 | [20, 22, 70, 65, 40] | 70, 35, 20 | walking |
| [50, 60, 50, 55] | 42 | [50, 60, 50, 55, 42] | 40, 75, 22 | driving |
| [80, 90, 60, 62] | 48 | [80, 90, 60, 62, 48] | 50, 55, 70 | load |
| | | | | lifting |

On determining the work type, the processor 101 may generate the fatigue score using the assigned weights to each of the plurality of sensors and subsequent values of the physiological signals. First, the processor 101 determines a weighted fatigue for each of the plurality of sensors using the physiological signals and the weight. Thereafter, the processor 101 generates a fatigue score from the weighted fatigue of each of the plurality of sensors. For example, if the processor 101 is determining the work type for an activity driving, then the sensors placed on leg muscles of a driver may be assigned with higher weights compared to the other sensors placed on different parts of the driver. This is because the leg muscles of the driver are more strained than any other parts of the driver. In some embodiments, the processor 101, at periodic time intervals determines work type of the subject as the activity of the subject may change over the period of time. Thus, in order to assign the weight dynamically the processor determines work type at regular intervals upon detecting work type for the first time”. In some embodiments of the present disclosure, the computing unit 100 may comprise an alert system (not shown) for generating an alarm. The alarm may be generated if the fatigue score is substantially close to or greater than a predefined threshold fatigue score.

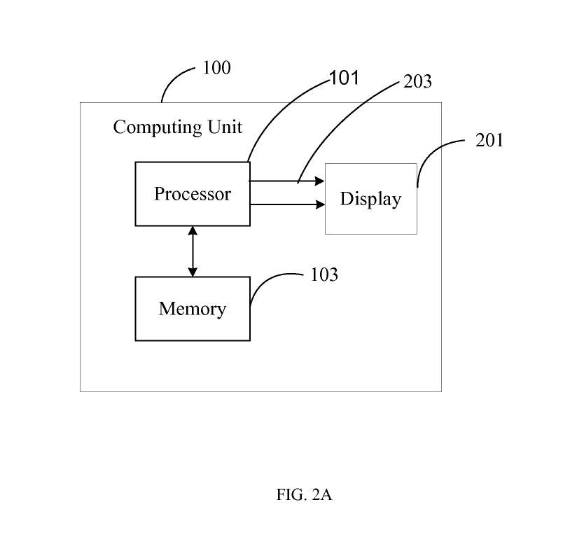

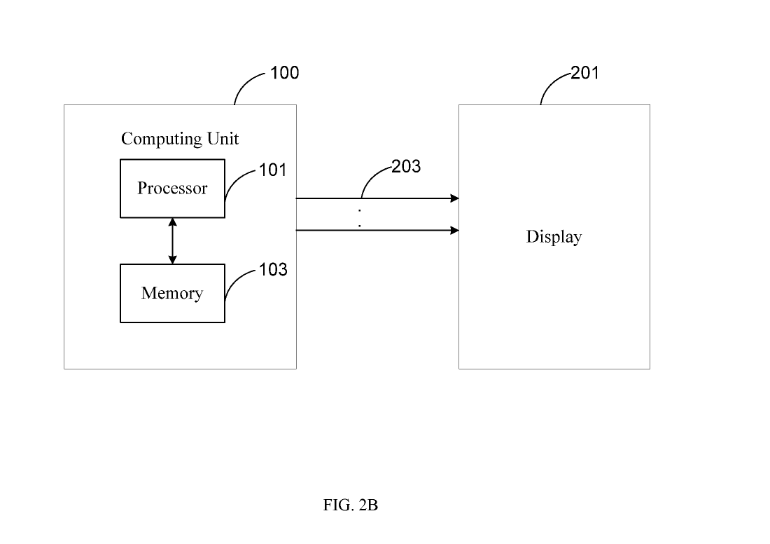

FIGS. 2A and 2B illustrates a computing unit 100 to monitor health condition of a subject and display the fatigue score on an associated display unit, in accordance with some embodiments of the present disclosure. In some embodiments, the computing unit may comprise at least one processor 101, a memory 103 storing instructions executable by the at least one processor 101 and a display unit 201 for displaying fatigue information 203 of at least one subject, as shown in FIG. 2A. In some embodiments, the computing unit may comprise at least one processor 101, a memory 103 storing instructions executable by the at least one processor 101 and an associated external display unit 201 for displaying fatigue information 203 of at least one subject, as shown in FIG. 2B. The computing unit 100 transmits fatigue information 203 such as, but not limited to, fatigue score, time for which the subject has performed a task for generating fatigue score and number of subjects for which fatigue score is generated, to the display unit 201. The display unit 201 displays the fatigue information, as received from the computing device 100. It will be apparent to a person skilled in the art that the display unit, including but not limited to, cathode ray tube display (CRT), Light-emitting diode display (LED), Plasma display panel (PDP), Liquid crystal display (LCD) and Organic light-emitting diode display (OLED) may be used.

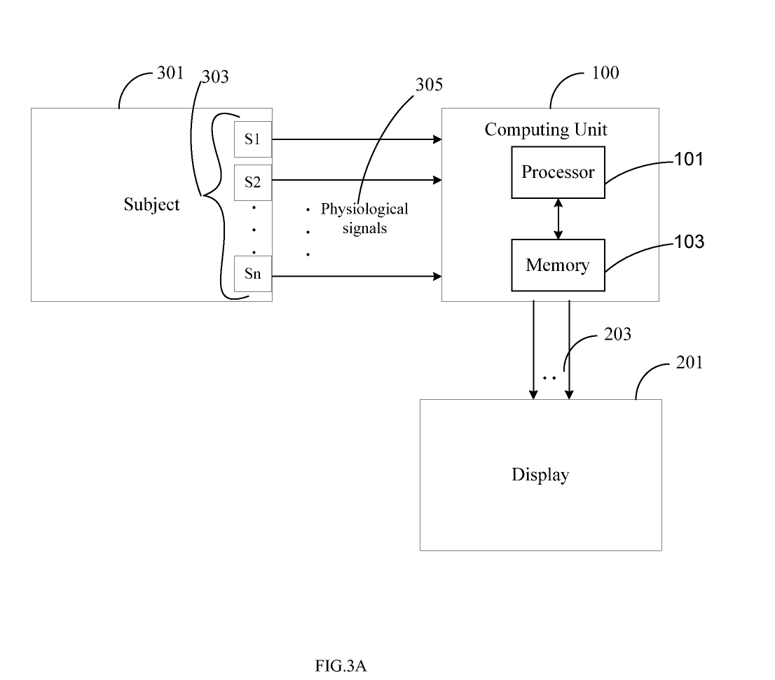

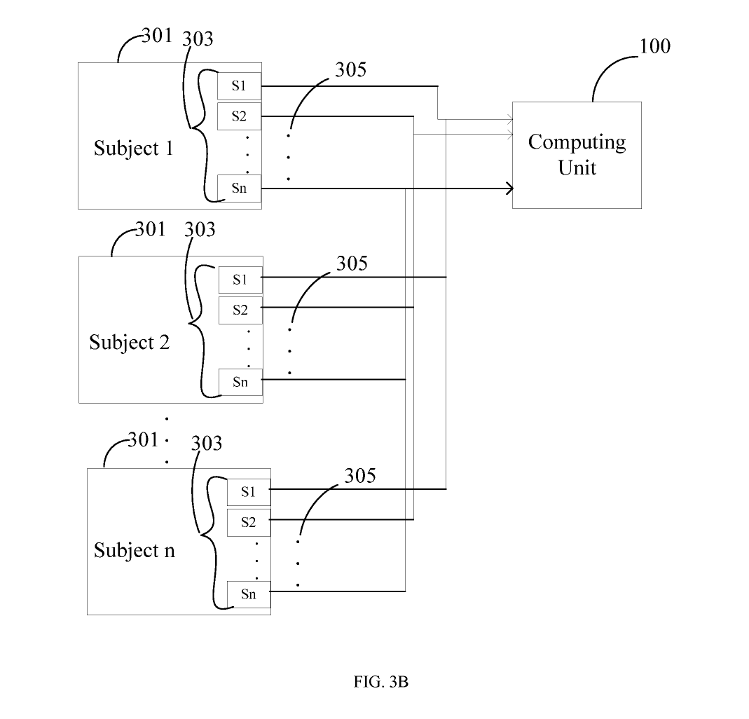

FIG. 3A illustrates an environment in which a computing unit 100 receives physiological signals associated with a subject in accordance with some embodiments of the present disclosure. The computing unit 100 may be configured to receive physiological signals 305 from a plurality of sensors (S1, S2 . . . Sn) 303 placed on the subject 301. The subject 301 may be one of a human being and an animal. The physiological signals may be received from plurality of sensors such as, but not limited to, at least one of Electrocardiograph (ECG) sensor, Electroencephalography (EEG) sensor, Electromyography (EMG) sensor and photo-plethysmo-graphy (PPG) sensor.

In an example embodiment, the sensors 303 may be placed in the form of adhesive patches on the body of a subject 301 such as, but not limited to a worker or an employee. For example, an ECG sensor may be placed on the upper center of the chest of the worker. In another example embodiment, a plurality of EMG sensors may be placed at plurality of locations on the body of the worker where muscle activation signal may be detected. The location of EMG sensors may depend on work type of the worker. As an example, for a driver the placement of sensors or sensor patches may be optimized to detect muscle activation related to driving such as the muscles of hands and legs of the driver. In another example, the location of sensors or sensor patches for mine workers may be optimized for detecting heavy physical work such as, but not limited to digging, carrying and loading.

The computing unit 100 may receive the physiological signals 305 from the plurality of sensors (S1, S2 . . . Sn) 303, using either wired or wireless means. In one exemplary embodiment, the computing unit may receive the physiological signals 305 using wireless radio technology such as, but not limited to, WiFi, Bluetooth and Zigbee. Further, the computing unit 100 may include a data aggregator for acquiring the physiological signals 305 from plurality of sensors 303. Upon acquiring the physiological signals 305 by the data aggregator, the signals are stored in the memory 103 for further processing.

The processor 101 may estimate a fatigue value from the physiological signals 305 using sensor specific methodology. The physiological signals 305 are received by the computing unit 100 from the plurality of sensors 303, wherein each sensor signals are converted into digital data and queued by the data aggregator. The queued data is processed using sensor specific method. In some exemplary embodiments, the processor 101 processes ECG signals from the ECG sensor using an ECG based fatigue estimation method. In another some exemplary embodiments, the processor 101 processes EMG signals from the EMG sensor using EMG-based fatigue estimation method. The processor 101 estimates the fatigue value from ECG signals upon calculating heart-rate (HR) of the subject. The HR is calculated by the processor 101 using one of R-peak detection method, artificial neural networks, genetic algorithms, wavelet transforms or filters Banks, Autocorrelation, ECG signal spectral analysis, trained regression model and any other machine learning method specific to ECG sensor. The processor 101 analyses the HR in frequency domain using FFT and maps ECG derived HR to fatigue level. Thus, the fatigue level is estimated by the processor 101.

In some embodiments, the processor 101 of the computing unit 100 uses frequency domain representation of the EMG signals using one of a regression model or any other machine learning method, for mapping the EMG frequency components to fatigue level. Similarly, in other embodiments, physiological data from other sensors attached to the subject may be mapped to the fatigue level using one of trained regression model and any other machine learning method. The estimated fatigue values are stored in the memory of the computing unit 101 for further processing.

The processor 101 may detect a work-type using the physiological signals 305. The processor 101 may extract physical signals information from all the physiological signals received from the plurality of sensors. For example, the processor 101 may extract frequency domain values for EMG signals only, out of all the extracted physical signals information. The processor 101 does not extract frequency domain values for other the physiological signals. The extracted frequency domain values of the EMG signals are compared or matched with reference models, wherein the reference models are known type of work activities. The reference models are activity-specific values which are generated from a reference data for each work type. The processor 101 compares the extracted frequency domain values from the EMG signals with the reference models using, one of nearest neighbor method such as, but not limited to Mahalanobis distance and Bhattacharya distance. Thus, the processor 101 identifies a matching reference value upon comparing the frequency domain values with a plurality of reference values. Thereafter, the processor 101 may identify a work type corresponding to the frequency domain value, which is substantially near to or equal to a matched reference value.

In some embodiments the processor 101 may dynamically assigns weights to the plurality of sensors based on the detected work-type. This is performed based on the following expression:

weight assigned to a sensor=K*activation energy of the sensor/C

where activation energy of the sensor is minimum electrical energy generated by the part of the body to the sensor, which the sensor can detect and produce an output accordingly. C is the sum of activation energies of all the sensors and K is a configurable gain parameter. The processor 101 generates activation energy of all the sensors from the physiological signals, preferably the EMG signals. The activation energy is obtained from the area under the power spectrum curve of the EMG signals. Thereafter, the processor 101 normalizes the weights of all the sensors such that, the sum of all sensor weights is equal to one. Also, the processor 101 assigns a constant weight to each of the plurality of sensors such as, but not limited to ECG sensor, EEG sensor and PPG sensor. The processor 101 dynamically assigns weights to the EMG sensor. The reason for assigning weight dynamically to the EMG sensors, in other words not assigning constant weight to the EMG sensors is that the activation energy (muscle activation correlating with different work types) varies for each activity performed by the worker and also, the location where the EMG sensors are placed on the body of the subject is not fixed unlike other sensors ECG, PPG and EEG.

Upon detecting the work type and assigning the work type to each of the plurality of sensors, the processor 101 may generate a fatigue score using the fatigue value obtained from the physiological signals and the weight of the plurality of sensors. The fatigue score indicates the health condition of the subject. After determining the health condition of the subject, the processor 101 may display the fatigue score on the associated display unit 203.

The processor 101 obtains modality-specific fatigue levels from regression models. The processor 101 uses a classifier algorithm such as support vector machine or any other machine learning method, which is trained for a specific sensor location on the subject and the work type. In some embodiment, for example, weights be w₁, w₂, w₃, . . . , w_{N} may be optimized for a predefined type of work or worker and sensor patch locations. The processor 101 generates an output by performing computations on the inputs as current weighted fatigue scores {w₁f₁, w₂f₂, w₃f₃ . . . w_{N}f_{N}} to generate an output fatigue score.

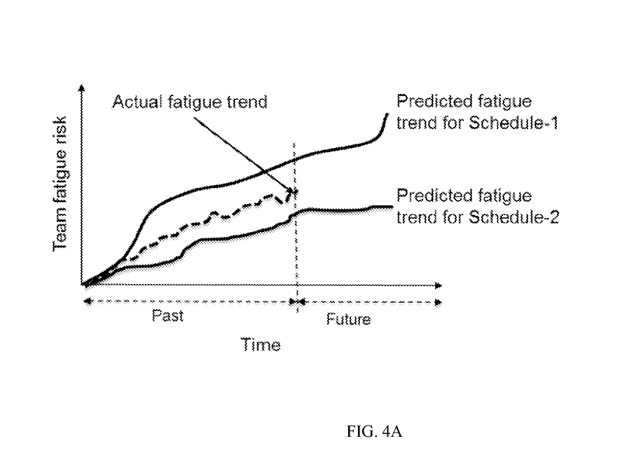

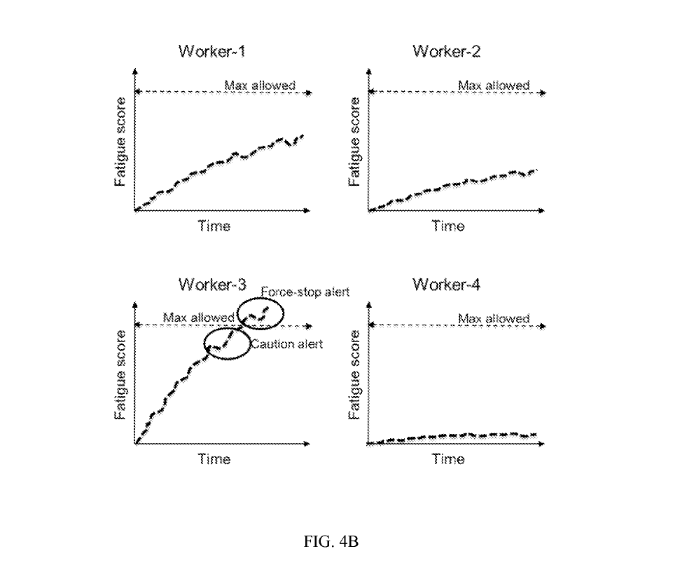

In some embodiments of the present disclosure, the computing unit 100 may generate a preventive alert and a team fatigue risk chart based on the fatigue scores of each subject or worker. The processor 101 of the computing unit 100 generates the preventive alerts and the estimates of the team fatigue risk at periodic intervals to produce the team risk fatigue chart. The processor 101 may initiate an alert signal, which may be broadcasted to all the workers, if the workers are about to reach the maximum allowed fatigue score or a threshold fatigue score. For example, the alert may be classified as ‘caution’ and ‘force-stop’, which corresponds to the fatigue score nearing maximum fatigue score and exceeding maximum fatigue score respectively. In some another embodiments, the processor 101 may send instructions to the display unit 203 for displaying the team risk fatigue chart as shown in FIG. 4A.

In some embodiments of the present disclosure, the processor 101 of the computing unit 100 may generate normalized fatigue unit (NFU). The NFU depends on a work-type performed by the worker. The NFU is defined as the increase in the fatigue score after performing a given job. The NFU includes physical and mental components. In some embodiments, an NFU is represented with a two element row vector NFU=[m, p]. For example, is a person attends a meeting for 30 minutes may cost NFU=[50, 10], where 50 is mental component units and 10 is physical component units. In another example, a task of ‘loading’ performed by a mine worker may cost NFU=[10, 90], in which the physical activity involved is higher than mental activity. The NFU for each job or work-type may be calculated from training data.

In some another embodiments of the present disclosure, the processor 101 of the computing unit 100 may generate a predictive job fatigue score (PJFS). The processor 101 may calculate for each worker, PJFS which is an increase in the physical and mental fatigue score in terms of NFU with respect to a specific job. For example, let a worker's current NFU is [10, 50], and attending a meeting may cost an NFU of [50, 15], then the predictive job fatigue score is [10+50, 50+15]=[60, 65] NFU.

In some another embodiments of the present disclosure, the processor 101 of the computing unit 100 may generate Team fatigue risk (TFR). The processor 101 may calculate team fatigue risk for N non-resting workers using the equation

TFR=[PJFS(w₁,jₐ)+PJFS(w₂,j_{b})+PJFS(w₃,j_{c})+ . . . PJFS(w_{N},j_{z})]/N

where w₁, w₂ . . . w_{z} denotes workers or subjects and jₐ, j_{b}, j_{c} . . . j_{c} denotes the work-type or job performed by the workers w₁, w₂ . . . w_{z}. The TFR denotes the predictive fatigue risk for a team for the selected worker job assignments based on different schedules in the work-type. In some embodiments, repeated assessment of the team fatigue risk is used to generate the team fatigue risk chart as shown in FIG. 4A. Using the trend in the team fatigue risk chart, the supervisor of the team may change or reschedule the worker-job assignments for the workers. The FIG. 4A illustrates a fatigue chart for plurality of subjects displayed on an associated display of the computing unit in accordance with some embodiment of the present disclosure.

FIG. 3B illustrates an environment in which a computing unit 100 receives physiological signals 305 from a plurality of subjects (subject 1, subject 2 . . . subject n) 301 in accordance with some embodiments of the present disclosure. The computing unit 100 includes at least one processor 101 and a memory 103 storing instructions executable by the at least one processor 101. Initially, the processor 101 may detect a work type of each subject based on the physiological signals received from said subject. Also, the processor 101 may assign a weight to each of the plurality of sensors based on the work type. Thereafter, the processor 101 may generate a fatigue score using the physiological signals and the weight of the plurality of sensors. The fatigue score indicates the health condition of the subject.

In some embodiments, the processor 101 may generate the team fatigue score using the fatigue score of all the plurality of subjects 301. The computing unit may further comprise an alert system for generating an alarm. Also, the exemplary computing unit 100 to monitor health condition of plurality of subjects 301 may be connected to a display (not shown in the FIG. 3B) for displaying a team's fatigue information. Here, the team means the plurality of subjects, whose health condition is monitored by the computing unit 100. In some embodiment, the fatigue information of each subject or worker may be displayed on a display 201 by the computing unit as shown in FIG. 4B. The FIG. 4B illustrates fatigue chart of plurality of workers separately, displayed on an associated display of the computing unit 100 in accordance with some embodiments of the present disclosure.

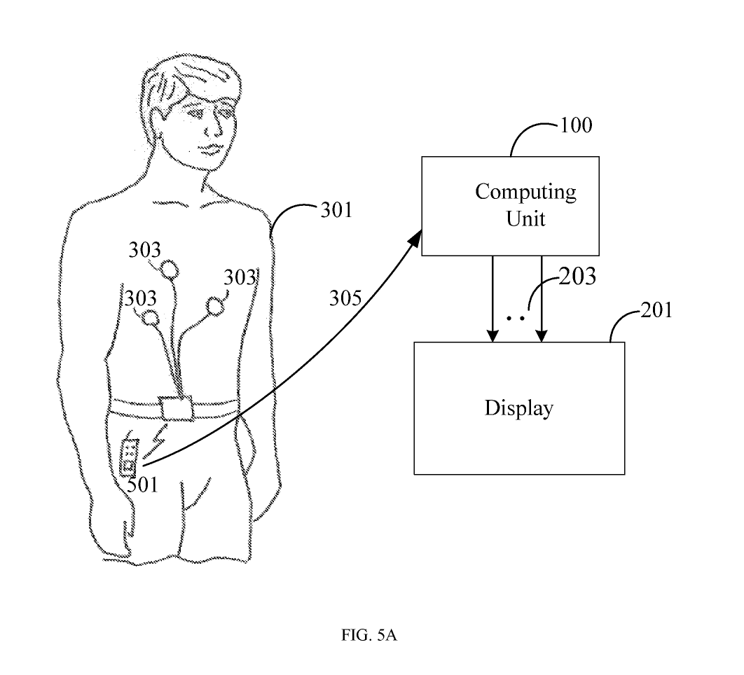

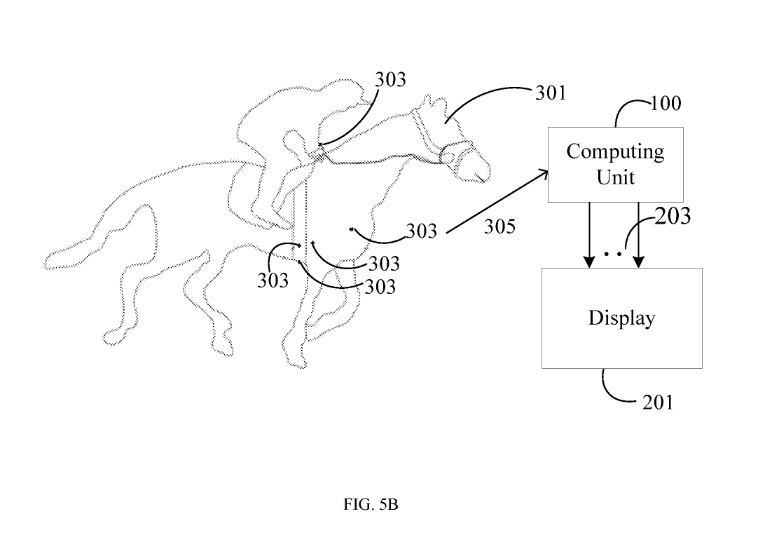

FIG. 5A illustrates an exemplary computing unit 100 to monitor health condition of a human 301 along with an associated display 201 in accordance with an example embodiment of the present disclosure. The computing unit 100 may be configured to receive physiological signals 305 from a plurality of sensors (S1, S2 . . . Sn) 303 placed on the human 301. In some example embodiments, the physiological signals may be transmitted by a transmitter 501 placed along with the plurality of sensors, on the body of human 301.

FIG. 5B illustrates an exemplary computing unit 100 to monitor health condition of an animal 301 along with an associated display 201 in accordance with another example embodiment of the present disclosure. The computing unit 100 may be configured to receive physiological signals 305 from a plurality of sensors (S1, S2 . . . Sn) 303 placed on the animal 301.

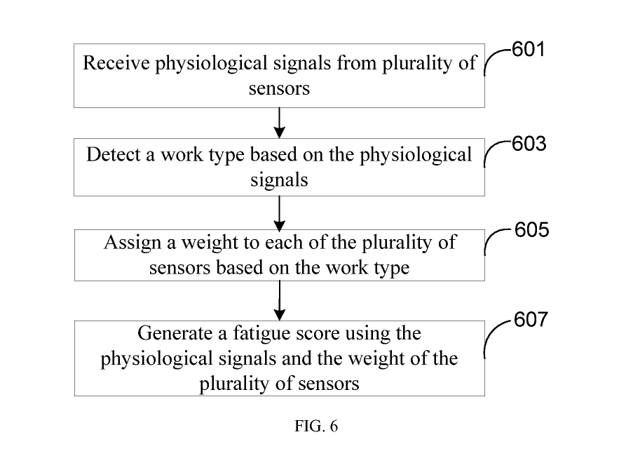

FIG. 6 shows a flowchart illustrating a method for monitoring health condition of a subject using a computing device in accordance with some embodiments of the present disclosure. At step 501, the computing unit may receive physiological signals from a plurality of sensors placed on the subject. Each of the plurality of sensors are placed on the subject at a location selected from at least one of head, muscles of arms, muscles of legs, scalp, sternum, midaxillary line, anterior axillary line, ear lobes and finger tips. The subject may be one of human and animal. The physiological signals are at least one of Electrocardiography (ECG) signal, Electroencephalography (EEG) signal, Electromyography (EMG) signal and photo-plethysmo-graphy (PPG) signal. At step 503, the computing unit may detect a work-type based on the physiological signals from the plurality of sensors. The computing unit may extract frequency domain values from the physiological signals. Next, the computing unit compares the extracted frequency domain values with a plurality of predefined reference values to identify matching reference value. Thereafter, the computing unit may identify a work type corresponding to the frequency domain value, which is substantially near to or equal to the matched reference value. At step 505, the computing unit may assign a weight to each of the plurality of sensors based on the work-type. At step 507, the computing unit may generate a fatigue score using the physiological signals and the weight of the plurality of sensors. The fatigue score indicates the health condition of the subject.

The described operations may be implemented as a method, system or article of manufacture using standard programming and/or engineering techniques to produce software, firmware, hardware, or any combination thereof. The described operations may be implemented as code maintained in a “non-transitory computer readable medium”, where a processor may read and execute the code from the computer readable medium. The processor is at least one of a microprocessor and a processor capable of processing and executing the queries. A non-transitory computer readable medium may comprise media such as magnetic storage medium (e.g., hard disk drives, floppy disks, tape, etc.), optical storage (CD-ROMs, DVDs, optical disks, etc.), volatile and non-volatile memory devices (e.g., EEPROMs, ROMs, PROMs, RAMs, DRAMs, SRAMs, Flash Memory, firmware, programmable logic, etc.), etc. The non-transitory computer-readable media comprise all computer-readable media except for a transitory. The code implementing the described operations may further be implemented in hardware logic (e.g., an integrated circuit chip, Programmable Gate Array (PGA), Application Specific Integrated Circuit (ASIC), etc.).

Still further, the code implementing the described operations may be implemented in “transmission signals”, where transmission signals may propagate through space or through a transmission media, such as an optical fiber, copper wire, etc. The transmission signals in which the code or logic is encoded may further comprise a wireless signal, satellite transmission, radio waves, infrared signals, Bluetooth, etc. The transmission signals in which the code or logic is encoded is capable of being transmitted by a transmitting station and received by a receiving station, where the code or logic encoded in the transmission signal may be decoded and stored in hardware or a non-transitory computer readable medium at the receiving and transmitting stations or devices. An “article of manufacture” comprises non-transitory computer readable medium, hardware logic, and/or transmission signals in which code may be implemented. A device in which the code implementing the described embodiments of operations is encoded may comprise a computer readable medium or hardware logic. Of course, those skilled in the art will recognize that many modifications may be made to this configuration without departing from the scope of the invention, and that the article of manufacture may comprise suitable information bearing medium known in the art.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required. On the contrary a variety of optional components are described to illustrate the wide variety of possible embodiments of the invention.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated operations of FIG. 6 show certain events occurring in a certain order. In alternative embodiments, certain operations may be performed in a different order, modified or removed. Moreover, steps may be added to the above described logic and still conform to the described embodiments. Further, operations described herein may occur sequentially or certain operations may be processed in parallel. Yet further, operations may be performed by a single processor or by distributed processing units.

The foregoing description of various embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims hereinafter appended.

Additionally, advantages of present disclosure are illustrated herein.

The present disclosure provides a method for monitoring health condition of a subject using a computing unit. The present disclosure enables a supervisor or a manager to take preventive measure upon detecting fatigue with respect to the current work-type and avoid unavoidable results. Also, the fatigue detect for monitoring health condition of a subject is applicable to one of humans and animals. Thus, this method may be customized to any work-type unlike methods which are specific to drivers or miner workers. Further, wearable sensors are attached to the subject's or worker's body and therefore do not limit the worker's presence based on constraints such as camera field of view. Thus, a worker working anywhere may be monitored using this method.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed:

1. A method for monitoring health condition of a subject, the method comprising:
receiving, by a health monitoring computing device, physiological signals from a plurality of sensors placed on the subject;
detecting, by the health monitoring computing device, a work-type based on the physiological signals from the plurality of sensors;
assigning, by the health monitoring computing device, a weight to each of the plurality of sensors based on the work-type; and
generating, by the health monitoring computing device, a fatigue score using the physiological signals and the weight of the plurality of sensors, wherein the fatigue score indicates the health condition of the subject.

2. The method as claimed in claim 1, wherein the subject is one of human being and animal.

3. The method as claimed in claim 1, wherein the physiological signals are at least one of Electrocardiography (ECG) signal, Electroencephalography (EEG) signal, Electromyography (EMG) signal and photo-plethysmo-graphy (PPG) signal.

4. The method as claimed in claim 1, wherein each of the plurality of sensors are placed on the subject at a location selected from at least one of head, muscles of arms, muscles of legs, scalp, sternum, midaxillary line, anterior axillary line, ear lobes or finger tips.

5. The method as claimed in claim 1, wherein the detecting of the work-type comprising:
extracting, by the health monitoring computing device, frequency domain values from the physiological signals;
comparing, by the health monitoring computing device, the frequency domain values with a plurality of predefined reference values to identify matching reference value; and
identifying, by the health monitoring computing device, a work type corresponding to the frequency domain value, which is substantially near to or equal to matched reference value.

6. The method as claimed in claim 1, wherein the generating the fatigue score comprising:
determining, by the health monitoring computing device, weighted fatigue for each of the plurality of sensors using the physiological signals and the weight; and
generating, by the health monitoring computing device, a fatigue score from the weighted fatigue of each of the plurality of sensors.

7. The method as claimed in claim 1, wherein the fatigue score is one of single value and multi-dimensional vector quantity.

8. The method as claimed in claim 1 further comprising generating, by the health monitoring computing device, an alarm if the fatigue score is substantially near to or greater than a predefined threshold fatigue score.

9. The method as claimed in claim 1 further comprising displaying, by the health monitoring computing device, the fatigue score on a display unit associated to the computing unit.

10. A health monitoring computing device comprising:
a processor;
a memory, wherein the memory coupled to the processor which are configured to execute programmed instructions stored in the memory comprising:
receiving physiological signals from a plurality of sensors placed on the subject;
detecting a work type based on the physiological signals;
assigning a weight to each of the plurality of sensors based on the work type; and
generating a fatigue score using the physiological signals and the weight of the plurality of sensors, wherein the fatigue score indicates the health condition of the subject.

11. The device as claimed in claim 10, wherein the sensors are at least one of Electrocardiograph (ECG) sensor, Electroencephalography (EEG) sensor, Electromyography (EMG) sensor and photo-plethysmo-graphy (PPG) signal.

12. The device as claimed in claim 10, wherein the processor is further configured to execute programmed instructions stored in the memory for the detecting further comprises:
extracting frequency domain values from the physiological signals;
comparing the frequency domain values with a plurality of predefined reference values to identify matching reference value; and
identifying a work type corresponding to the frequency domain value, which is substantially near to or equal to matched reference value.

13. The device as claimed in claim 10, wherein the processor is further configured to execute programmed instructions stored in the memory for the generating the fatigue score:
determining weighted fatigue for each of the plurality of sensors using the physiological signals and the weight; and
generating a fatigue score from the weighted fatigue of each of the plurality of sensors.

14. The device as claimed in claim 10, wherein the processor is further configured to execute programmed instructions stored in the memory further comprising generating an alarm if the fatigue score is substantially near to or greater than a predefined threshold fatigue score.

15. The device as claimed in claim 10, wherein the processor is further configured to execute programmed instructions stored in the memory further comprising displaying the fatigue score on a display unit associated to the computing unit.

16. A non-transitory computer readable medium having stored thereon instructions for monitoring health condition of a subject comprising executable code which when executed by a processor, causes the processor to perform steps comprising:
receiving physiological signals from a plurality of sensors placed on the subject;
detecting a work-type based on the physiological signals;
assigning a weight to each of the plurality of sensors based on the work-type; and
generating a fatigue score using the physiological signals and the weight of the plurality of sensors, wherein the fatigue score indicates the health condition of the subject.

17. The medium as claimed in claim 16, wherein the instructions further cause the at least one processor to perform the detecting the work type comprising:
extracting frequency domain values from the physiological signals;
comparing the frequency domain values with a plurality of predefined reference values to identify matching reference value; and
identifying a work type corresponding to the frequency domain value, which is substantially near to or equal to matched reference value.

18. The medium as claimed in claim 16, wherein the instructions further cause the at least one processor to perform the generating the fatigue score comprising:
determining weighted fatigue for each of the plurality of sensors using the physiological signals and the weight; and
generating a fatigue score from the weighted fatigue of each of the plurality of sensors.
